(19) United States
(12) Patent Application Publication
(10) Pub. No.: US 2026/0048152 A1
(43) Pub. Date: Feb. 19, 2026

(54) GEMCITABINE INORGANIC-ORGANIC HYBRID NANOPARTICLES

(71) Applicants: Karlsruher Institut für Technologie Körperschaft des öffentlichen Rechts, Karlsruhe (DE); Georg-August-Universität Göttingen Stiftung Öffentlichen Rechts, Universitätsmedizin, Göttingen (DE); Max-Planck-Gesellschaft zur Förderung der Wissenschaften e.V., München (DE)

(72) Inventors: Claus Feldmann, Ettlingen (DE); Kristina Posselt, Weingarten (DE); Christof Niemeyer, Bremen (DE); Leonie Schneider, Bad Säckingen (DE); Frauke Alves, Göttingen (DE); Joanna Napp, Rosdorf (DE); Myrto Ischyropoulou, Veksoe (DK)

(21) Appl. No.: 18/802,004

(22) Filed: Aug. 13, 2024

(51) Int. Cl. A61K 49/00; A61K 9/51; A61K 31/7068; A61K 47/68

(52) U.S. Cl. CPC A61K 49/005; A61K 9/5115; A61K 31/7068; A61K 47/6849

(57) ABSTRACT

The present invention relates to an inorganic-organic hybrid compound as ionic compound, composed of an inorganic metal cation selected from [ZrO]²⁺, and of an organic active ingredient anion selected from gemcitabine monophosphate or gemcitabine triphosphate.

### FIELD OF THE INVENTION

The present invention relates to an inorganic-organic hybrid compound as ionic compound, comprising the cation [ZrO]²⁺and an organic active ingredient anion selected from gemcitabine monophosphate or gemcitabine triphosphate.

### BACKGROUND

Pancreatic ductal adenocarcinoma (PDAC) is one of the most lethal human cancers, ranking the fourth leading cause of cancer-related deaths in the western world. Such dismal prognosis can be traced back to both its late detection—often due to a lack of symptoms in early stages—and to treatment failure—especially in advanced tumors not eligible for complete surgical resection. Therefore, effective therapeutic strategies not only against the primary tumor but also its metastasis (most often in liver, peritoneum and lung) are urgently needed for patients with late stage PDAC. Currently, systemic chemotherapy represents the standard treatment either in neo-adjuvant, adjuvant and palliative settings, regrettably resulting in only modest improvement of survival. Since the late 1990s, the nucleoside analogue gemcitabine (GEM, 2′,2′-difluroro-2′-deoxycytidine) has been the first-line drug for advanced PDAC. GEM is preferentially applied in combination therapies (e.g., with nab-paclitaxel, cisplatin, capecitabine, erlotinib) selected based on the tumor stage and the general performance status. Although other regimens are available (e.g. FOLFIRINOX: combination of 5-ﬂuorouracil, leucovorin, irinotecan, oxaliplatin), GEM is still a key-compound for PDAC treatment. Yet, its clinical efficacy is severely limited by its short half-life and rapid elimination from the body by conversion into inactive 2′,2′-difluorodeoxyuridine by the enzyme cytidine deaminase (CDA) abundant in plasma and liver. Therefore, usually high GEM doses need to be repeatedly given to patients, which not only lead to systemic toxicity (e.g., hepato-/nephrotoxicity) but also promotes chemoresistance.

Since GEM is a hydrophilic moiety, it needs to be transported into the tumor cells via different nucleoside transporters, e.g., the human equilibrative nucleoside transporter (hENT1), whose biological activity is considered to be a prerequisite for the efficacy of GEM. After entering the cell, free GEM undergoes intracellular conversion by a series of phosphorylation steps mediated by specific enzymes. First of all, deoxycytidine kinase (dCK) phosphorylates GEM to the nucleotide gemcitabine monophosphate (GMP), and subsequently pyrimidine nucleoside monophosphate kinase (NMPK) to gemcitabine diphosphate. The latter is finally activated by phosphorylation to gemcitabine triphosphate (GTP) by the nucleoside diphosphate kinase (NDPK) that competes with deoxycytidine triphosphate (dCTP) for the incorporation into DNA, leading to an inhibition of DNA synthesis.

The most fundamental mechanisms of GEM chemoresistance are: i) down-regulation of the nucleoside transporters, as their absence prevents GEM from entering cells, ii) decrease in expression of dCK being responsible for the first step of the GEM phosphorylation after cellular uptake, and iii) enhanced GEM deactivation, causing GEM degradation and excretion, controlled by the enzyme cytidine deaminase, which is often up-regulated in chemoresistant patients. Another factor generally affecting the efficacy of chemotherapeutic drugs is the peculiar tumor microenvironment in PDAC with a highly dense, desmoplastic stroma. To circumvent the resistance to GEM derived from the physical and molecular barriers in PDAC, nanocarrier-based approaches (e.g. GEM-loaded solid-lipid nanocarriers, GEM embedded in (bio)polymer matrices or liposomes) (see a) Y. Yao, Y. Zhou, L. Liu, Y. Xu, Q. Chen, Y. Wang, S. Wu, Y. Deng, J. Zhang, A Shao, Front. Mol. Biosci. 2020, 7, 193. b) G. Mondal, S. Almawash, A. K. Chaudhary, R. I. Mahato, Mol. Pharm. 2017, 14, 3121-3133. c) T. -J. Liang, Z. -M. Zhou, Y. -Q. Cao, M. -Z. Ma, X. -J. Wang, K. Jing, Internat. J. Pharm. 2016, 513, 564-571. d) K. K. Frese, A. Neesse, N. Cook, T. E. Bapiro, M. P. Lolkema, D. I. Jodrell, D. A. Tuveson, Cancer Discov. 2012, 2, 260-269. e) C. Celia, D. Cosco, D. Paolino, M. Fresta, Expert Opin. Drug Deliv. 2011, 8, 1609-1629) and various modifications of GEM (e.g. as prodrug) (see a) K. O. Affram, T. Smith, E. Ofori, S. Krishnan, P. Underwood, J. G. Trevino, E. Agyare, J. Drug Deliv. Sci. Technol. 2020, 55, 101374. b) K Samanta, S. Setua, S. Kumari, M. Jaggi, M. M. Yallapu, S. C. Chauhan, Pharmaceut. 2019, 11, 574) have been suggested but yet suffer from insufficient drug load (<10 % of total nanocarrier mass), multi-step synthesis, complex nanocarrier composition and structure, and/or insufficient drug transport, delivery and efficacy. A nanocarrier-based transport of GMP, however, could not only be an option to bypass the hENT1 transporter but also the initial step of GEM phosphorylation as the rate-limiting step to activate gemcitabine. Both strategies have high potential to circumvent GEM resistance as already suggested for GEM-loaded liposomes, microemulsion-made calcium phosphate nanocarriers, or GMP-filled MOF-type particles. However, these nanocarriers exhibit complex, multicomponent compositions with only <1 to 10 % of GEM/GMP and particle sizes partly up to 100 μm.

Numerous pathways were described for the internalization of nanocarrier-based therapeutics into cells. After uptake, these pathways merge into early endosomes before undergoing sorting, where material may also be sent back to the surface via recycling endosomes or traffic to late endosomes and lysosomes. After uptake, these pathways merge into early endosomes before undergoing sorting, where material may also be sent back to the surface via recycling endosomes, traffic to late endosomes and lysosomes or escape from endocytic vesicles to the cytosol. Understanding the complex mechanisms and pathways, which can be different for different cell types as well as for different nanocarriers, is a currently emerging issue. Metabolic characteristics of cancer cells such as pH or increased glucose consumption can be used to direct nanocarriers to the tumor tissue.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure.

Aiming at an efficient delivery of GEM to PDAC tumors, preferentially circumventing chemoresistance, the inventors suggest inorganic-organic hybrid nanoparticles (IOH-NPs) as a novel drug-delivery system for selective delivery of extraordinarily high concentrations of already phosphorylated gemcitabine monophosphate (GMP) to the primary tumor and metastatic sites, followed by uptake into tumor cells, most likely via one of the endocytic pathways but independent of the activity of hENT1. Due to fluorescence labelling, the GMP-loaded IOH-NPs (GMP-IOH-NPs) are enabled for in vivo and ex vivo monitoring. Moreover, “naked” GMP-IOH-NPs are compared with glucose-coated (GMP-GLU-IOH-NPs) and cetuximab-functionalized (GMP-CTX-IOH-NPs) in regard of an efficient, selective accumulation at tumor sites and lesions. IOH-NP accumulation, tumor targeting, cytostatic efficacy, chemoresistance, and side effects are addressed to understand and to maximize the therapeutic benefits applying GMP-IOH-NPs.

Accordingly, the present invention provides an inorganic-organic hybrid compound as ionic compound, comprising the cation [ZrO]²⁺and an organic active ingredient anion selected from gemcitabine monophosphate or gemcitabine triphosphate.

In one embodiment, the inorganic-organic hybrid compound has a particle diameter in the range from 1 to 100 nm, i.e. is present in the form of nanoparticles (GMP-IOH-NPs).

The compound in accordance with the invention represents a novel drug-delivery system for the selective delivery of extraordinarily high concentrations of gemcitabine monophosphate (GMP) or gemcitabine triphosphate (GTP) not only to the primary tumor but also to metastatic sites. GMP-IOH-NPs have a composition [ZrO]²⁺[GMP]²⁻ with GMP as drug anion (76% of total IOH-NP mass). Multiscale fluorescence imaging confirms an efficient uptake in tumor cells, independent of the activity of the human-equilibrative-nucleoside transporter (hENT1), being responsible for gemcitabine (GEM) transport into cells and a key factor for GEM resistance. Delivering already phosphorylated GMP via GMP-IOH-NPs into tumor cells also allows to overcome cellular resistance induced by the down-regulation of deoxycytidine kinase. GMP-IOH-NPs show high accumulation in tumor lesions and only minor liver trapping when given intraperitoneally. GMP-IOH-NPs result in a higher anti-tumor efficacy compared to free GEM and is further enhanced applying cetuximab-functionalized GMP-CTX-IOH-NPs. By maximizing the therapeutic benefits with high drug load, tumor-specific delivery, minimizing undesired side effects, overcoming mechanisms of chemoresistance, and preventing systemic GEM inactivation, GMP-IOH-NPs are considered to have a high chance to significantly improve current PDAC-patient outcome.

In one embodiment, the inorganic-organic hybrid compound is [ZrO]²⁺[GMP]²⁻ or [ZrO]²⁺₃[GTP]³⁻₂.

In one embodiment, the inorganic-organic hybrid compound is in X-ray-amorphous form.

In another embodiment, the inorganic-organic hybrid compound further comprises a fluorescent dye anion which carries a phosphate, phosphonate, sulfate, sulfonate, carbonate or carboxylate group as functional group.

In yet another embodiment, the inorganic-organic hybrid compound is further functionalized with an antibody, peptide or oligonucleotide, particularly cetuximab-functionalized.

In still another embodiment, the inorganic-organic hybrid compound is glucose-coated.

In one aspect, provided is an inorganic-organic hybrid compound as ionic compound, comprising the cation [ZrO]²⁺ and an organic active ingredient anion selected from gemcitabine monophosphate or gemcitabine triphosphate.

In one embodiment the inorganic-organic hybrid has a particle diameter in the range from 1 to 100 nm.

In one embodiment the inorganic-organic hybrid is [ZrO]²⁺[GMP]²⁻ or [ZrO]²⁺₃[GTP]³⁻₂.

In one embodiment the inorganic-organic hybrid is in X-ray-amorphous form.

In one embodiment the inorganic-organic hybrid further comprises a fluorescent dye anion which carries a phosphate, phosphonate, sulfate, sulfonate, carbonate, or carboxylate group as functional group.

In one embodiment, the organic fluorescent dye anion is derived from fluorescent dyes selected from the group consisting of 1,1′-diethyl-2,2′-cyanine iodide, 1,2-diphenylacetylene, 1,4-diphenylbutadiene, 1,6-diphenylhexatriene, 2,5-diphenyloxazole, 2-methylbenzoxazole, 4′,6-diamidino-2-phenylindole (DAPI), 4-(dicyanomethylene)-2-methyl-6-(p-dimethylaminostyryl)-4H-pyran (DCM), 4-dimethylamino-4′-nitrostilbene, 5,10,15-triphenylcorrole, 5,10,15-tris(pentafluorophenyl)corrole, 5,10-diarylchlorin, 5,10-diarylcopper chlorin, 5,10-diarylcopper oxochlorin, 5,10-diarylmagnesium oxochlorin, 5,10-diaryloxochlorin, 5,10-diarylzinc chlorin, 5,10-diarylzinc oxochlorin, 7-benzylamino-4-nitrobenz-2-oxa-1,3-diazole, 7-methoxycoumarin-4-acetic acid, 9,10-bis(phenylethynyl)anthracene, 9,10-diphenylanthracene, acridine orange, acridine yellow, adenine, anthracene, anthraquinone, auramine O, azobenzene, bacteriochlorophyll A, benzoquinone, beta-carotene, bilirubin, biliverdin dimethyl ester, biphenyl, bis(5-mesityldipyrrinato)zinc, bis(5-phenyldipyrrinato)zinc, boron subphthalocyanine chloride, chlorin E6, chlorophyll A, chlorophyll B, cis-stilbene, coumarin and its derivatives, cresyl violet perchlorate, cryptocyanine, crystal violet, cytosine, dansylglycine, diprotonated tetraphenylporphyrin, eosine and its derivatives, ethyl (p-dimethylamino)benzoate, ferrocene, fluorescein and its derivatives, as for example methylfluorescein, resorufin, amaranth, aluminum(III)-phthalocyanine chloride tetrasulfonic acid, trypan blue, guanine, hematin, histidine, Hoechst 33258, indocarbocyanine and its derivatives, lucifer yellow CH, magnesium octaethylporphyrin, magnesium phthalocyanine, magnesium tetramesitylporphyrin, magnesium tetraphenylporphyrin, malachite green, merocyanine, N, N′-difluoroboryl-1,9-dimethyl-5-(4-iodophenyl)dipyrrin, N,N′-difluoroboryl-1,9-dimethyl-5-[(4-(2-trimethylsilylethynyl), N,N′-difluoroboryl-1,9-dimethyl-5-phenyldipyrrin, tetraphenylporphyrin, naphthalene, nile blue, nile red, octaethylporphyrin, oxacarbocyanine and its derivatives, oxazine and its derivatives, p-quaterphenyl, p-terphenyl, perylene and its derivatives, phenol, phenylalanine, phenyldipyrrin, pheophorbide, phthalocyanine, pinacyanol iodide, piroxicam, porphin, proflavin, protoporphyrin IX dimethyl ester, pyrene, pyropheophorbide and its derivatives, pyrrol, quinine, rhodamine and its derivatives, riboflavin, bengal red, squarylium dye III, TBP beta-octa(COOBu)-Fb, TBP beta-octa(COOBu)-Pd, TBP beta-octa(COOBu)-Zn, TBP meso-tetraphenyl-beta-octa(COOMe)-Fb, TBP meso-tetraphenyl-beta-octa(COOMe)-Pd, TBP meso-tetraphenyl-beta-octa(COOMe)-Zn, TCPH meso-tetra(4-COOMe-phenyl)-Fb, TCPH meso-tetra(4-COOMe-phenyl)-Pd, TCPH meso-tetra(4-COOMe-phenyl)-Zn, tetra-tert-butylazaporphin, tetra-tert-butylnaphthalocyanine, tetrakis(2,6-dichlorophenyl)porphyrin, tetrakis(o-aminophenyl)porphyrin, tetramesitylporphyrin, tetraphenylporphyrin, tetraphenylsapphyrin, thiacarbocyanine and its derivatives, thymine, trans-stilbene, tris(2,2′-bipyridyl)ruthenium(II), tryptophan, thyrosine, uracil, vitamin B12, zinc octaethylporphyrin, phthalocyanine and its derivatives, porphyrin and its derivatives, including tetra(o-amidophosphonatophenyl)porphyrin, and umbelliferone, where the organic fluorescent dyes which do not as such have a phosphate, phosphonate, sulfate, sulfonate, carbonate, or carboxylate group have been modified with at least one of these functional groups.

In one embodiment the inorganic-organic hybrid is doped with a lanthanoid selected from Ce, Pr, Nd, Sm, Eu, Gd, Tb, Dy, Ho, Er, Tm, Yb, or Lu, with a transition metal selected from Cr, Mn, Cu, Zn, Y, Ag, or Cd, with a main group element selected from Sn, Sb, Pb, or Bi, or with a complex anion selected from [VO₄]³⁻, [MoO₄]³⁻or [WO₄]³⁻.

In one embodiment the inorganic-organic hybrid is further functionalized with an antibody, peptide or oligonucleotide.

In one embodiment the inorganic-organic hybrid is cetuximab-functionalized.

In one embodiment the inorganic-organic hybrid is glucose-coated.

In another aspect, provided is a method of treating pancreatic ductal adenocarcinoma (PDAC) by administering the inorganic-organic hybrid compound of claim 1.

In one embodiment of the method, the inorganic-organic hybrid compound has a particle diameter in the range from 1 to 100 nm.

In one embodiment of the method, the inorganic-organic hybrid compound is [ZrO]²⁺[GMP]²⁻ or [ZrO]²⁺₃[GTP]³⁻₂.

In one embodiment of the method, the inorganic-organic hybrid compound is in X-ray-amorphous form.

In one embodiment of the method, the inorganic-organic hybrid compound further comprises a fluorescent dye anion which carries a phosphate, phosphonate, sulfate, sulfonate, carbonate, or carboxylate group as functional group.

In one embodiment of the method, the fluorescent dye anion is derived from fluorescent dyes selected from the group consisting of 1,1′-diethyl-2,2′-cyanine iodide, 1,2-diphenylacetylene, 1,4-diphenylbutadiene, 1,6-diphenylhexatriene, 2,5-diphenyloxazole, 2-methylbenzoxazole, 4′,6-diamidino-2-phenylindole (DAPI), 4-(dicyanomethylene)-2-methyl-6-(p-dimethylaminostyryl)-4H-pyran (DCM), 4-dimethylamino-4′-nitrostilbene, 5,10,15-triphenylcorrole, 5,10,15-tris(pentafluorophenyl)corrole, 5,10-diarylchlorin, 5,10-diarylcopper chlorin, 5,10-diarylcopper oxochlorin, 5,10-diarylmagnesium oxochlorin, 5,10-diaryloxochlorin, 5,10-diarylzinc chlorin, 5,10-diarylzinc oxochlorin, 7-benzylamino-4-nitrobenz-2-oxa-1,3-diazole, 7-methoxycoumarin-4-acetic acid, 9,10-bis(phenylethynyl)anthracene, 9,10-diphenylanthracene, acridine orange, acridine yellow, adenine, anthracene, anthraquinone, auramine O, azobenzene, bacteriochlorophyll A, benzoquinone, beta-carotene, bilirubin, biliverdin dimethyl ester, biphenyl, bis(5-mesityldipyrrinato)zinc, bis(5-phenyldipyrrinato)zinc, boron subphthalocyanine chloride, chlorin E6, chlorophyll A, chlorophyll B, cis-stilbene, coumarin and its derivatives, cresyl violet perchlorate, cryptocyanine, crystal violet, cytosine, dansylglycine, diprotonated tetraphenylporphyrin, eosine and its derivatives, ethyl (p-dimethylamino)benzoate, ferrocene, fluorescein and its derivatives, as for example methylfluorescein, resorufin, amaranth, aluminum(III)-phthalocyanine chloride tetrasulfonic acid, trypan blue, guanine, hematin, histidine, Hoechst 33258, indocarbocyanine and its derivatives, lucifer yellow CH, magnesium octaethylporphyrin, magnesium phthalocyanine, magnesium tetramesitylporphyrin, magnesium tetraphenylporphyrin, malachite green, merocyanine, N, N′-difluoroboryl-1,9-dimethyl-5-(4-iodophenyl)dipyrrin, N,N′-difluoroboryl-1,9-dimethyl-5-[(4-(2-trimethylsilylethynyl), N,N′-difluoroboryl-1,9-dimethyl-5-phenyldipyrrin, tetraphenylporphyrin, naphthalene, nile blue, nile red, octaethylporphyrin, oxacarbocyanine and its derivatives, oxazine and its derivatives, p-quaterphenyl, p-terphenyl, perylene and its derivatives, phenol, phenylalanine, phenyldipyrrin, pheophorbide, phthalocyanine, pinacyanol iodide, piroxicam, porphin, proflavin, protoporphyrin IX dimethyl ester, pyrene, pyropheophorbide and its derivatives, pyrrol, quinine, rhodamine and its derivatives, riboflavin, bengal red, squarylium dye III, TBP beta-octa(COOBu)-Fb, TBP beta-octa(COOBu)-Pd, TBP beta-octa(COOBu)-Zn, TBP meso-tetraphenyl-beta-octa(COOMe)-Fb, TBP meso-tetraphenyl-beta-octa(COOMe)-Pd, TBP meso-tetraphenyl-beta-octa(COOMe)-Zn, TCPH meso-tetra(4-COOMe-phenyl)-Fb, TCPH meso-tetra(4-COOMe-phenyl)-Pd, TCPH meso-tetra(4-COOMe-phenyl)-Zn, tetra-tert-butylazaporphin, tetra-tert-butylnaphthalocyanine, tetrakis(2,6-dichlorophenyl)porphyrin, tetrakis(o-aminophenyl)porphyrin, tetramesitylporphyrin, tetraphenylporphyrin, tetraphenylsapphyrin, thiacarbocyanine and its derivatives, thymine, trans-stilbene, tris(2,2′-bipyridyl)ruthenium(II), tryptophan, thyrosine, uracil, vitamin B12, zinc octaethylporphyrin, phthalocyanine and its derivatives, porphyrin and its derivatives, including tetra(o-amidophosphonatophenyl)porphyrin, and umbelliferone, where the organic fluorescent dyes which do not as such have a phosphate, phosphonate, sulfate, sulfonate, carbonate, or carboxylate group have been modified with at least one of these functional groups.

In one embodiment of the method, the inorganic-organic hybrid compound is doped with a lanthanoid selected from Ce, Pr, Nd, Sm, Eu, Gd, Tb, Dy, Ho, Er, Tm, Yb, or Lu, with a transition metal selected from Cr, Mn, Cu, Zn, Y, Ag, or Cd, with a main group element selected from Sn, Sb, Pb, or Bi, or with a complex anion selected from [VO₄]³⁻, [MoO₄]³⁻ or [WO₄]³⁻.

In one embodiment of the method, the inorganic-organic hybrid compound is further functionalized with an antibody, peptide, or oligonucleotide.

In one embodiment of the method, the inorganic-organic hybrid compound is cetuximab-functionalized.

In one embodiment of the method, the inorganic-organic hybrid compound is glucose-coated.

### BRIEF DESCRIPTION OF THE DRAWINGS

FIG. 1 shows the synthesis and characterization of [ZrO]²⁺[GMP]²⁻ IOH-NPs: a) Scheme illustrating the aqueous synthesis, b) particle size distribution according to DLS and SEM with photo of aqueous suspension, c) SEM images at different levels of magnification, d) FT-IR spectra (with H2(GMP) as a reference), e) zeta potential, including surface-functionalized [ZrO]²⁺[GMP]²⁻@[ZrO]²⁺[G6P]²⁻ and [ZrO]²⁺[GMP]²⁻@CTX IOH-NPs (G6P: glucose-6-phosphate; CTX: cetuximab), f) scheme of IOH-NPs with designation used for in vitro/in vivo studies.

FIG. 2 shows a schematic representation of the protocol for establishing GEM-resistant PDAC cell-lines, here KPC and AsPC-1 cells. Initially, native KPC or AsPC-1 cells were cultured in a medium with stepwise increases in GEM (Gemcitabine) concentration during the adaptation phase, until resistance was achieved at a final concentration of 1000 nM (consolidation phase). After acquiring GEM resistance, the pancreatic ductal adenocarcinoma (PDAC) cells were maintained in a medium containing 700 nM GEM to ensure the stability of the GEM-resistant phenotype.

FIG. 3 shows the anti-tumor efficacy of GMP-IOH-NPs in GEM-Resistant KPC - PDAC cells in comparison to native KPC cells. Cell viability of KPC-Native cells (left graph) and KPC-Resistant cells (right graph) after 6 days of treatment with Free GEM (blue line), Ref-IOH-NP (red line), and GMP-IOH-NPs (green line). Cell viability was assessed using the luminescent CellTiter-Glo assay, and results are presented as the percentage of viable cells normalized to untreated (unstimulated) cells. Data is presented as mean±SEM. Two-way ANOVA, followed by Dunnett's multiple comparisons was performed from triplicates of three independent experiments (**p≤0.01).

FIG. 4 shows the anti-tumor efficacy of GMP-IOH-NPs and GTP-IOH-NPs in human GEM-Resistant AsPC-1 PDAC cells in comparison to Native AsPC-1 cells. Cell viability of AsPC-1 Native cells (upper graphs) after 6 days of treatment with Free GEM (blue line), GMP-IOH-NPs (green line, left graph) and GTP-IOH-NP (green line, right graph). Cell viability of AsPC-1 Resistant cells (lower graph) after 6 days in response to treatment with Free GEM (blue line), GMP-IOH-NPs (green line) and GTP-IOH-NP (red line). Cell viability was assessed using the luminescent CellTiter-Glo assay, and results are presented as the percentage of viable cells normalized to unstimulated (untreated) cells. Data is presented as mean±SEM. Two-way ANOVA, followed by Dunnett's multiple comparisons was performed from triplicates of three independent experiments. Statistical differences as free GEM vs. GMP-IOH-NP; free GEM vs. GTP-IOH-NP; and GMP-IOH-NP vs. GTP-IOH-NP are calculated from all values of all concentrations in the range of 1000 to 2000 nM (***p≤0.001, ****p<0.0001).

### DETAILED DESCRIPTION

Provided is an inorganic-organic hybrid compound as ionic compound, composed of an inorganic metal cation selected from [ZrO]²⁺, and of an organic active ingredient anion selected from gemcitabine monophosphate or gemcitabine triphosphate.

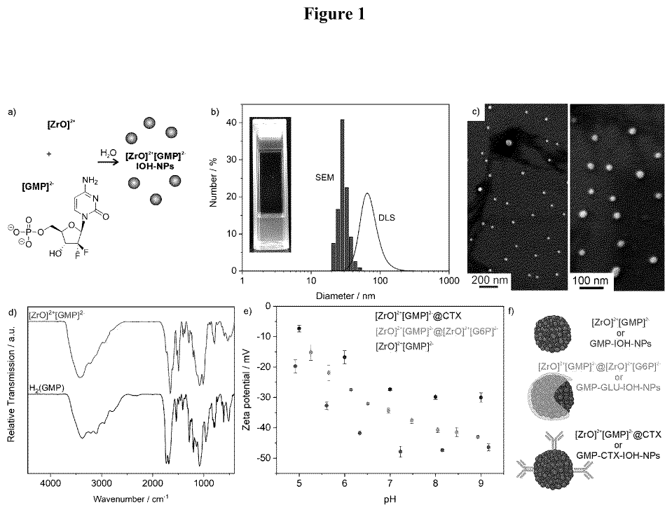

Inorganic-organic hybrid nanoparticles (IOH-NPs) are characterized by a saline composition with an inorganic cation and a drug anion, which is functionalized by phosphate, sulfonate, or carboxylate groups (see B. L. Neumeier, M. Khorenko, F. Alves, O. Goldmann, J. Napp, U. Schepers, H. M. Reichardt, C. Feldmann, ChemNanoMat 2019, 5, 24-45). Specifically, the concept of IOH-NPs comprises a simple synthesis in water, an extraordinary high drug load (>60 % of total nanoparticle mass), an uncomplex composition and structure of the nanocarriers, and a high adaptability of the IOH-NPs to use various drugs. Previous studies with antibiotic or anti-inflammatory drugs have already pointed to the feasibility of the material concept. Here, gemcitabine monophosphate (GMP) is used as chemotherapeutic anion for the first time. Zirconyl ([ZrO]²⁺) is used as inorganic cation to make the drug insoluble in water and to obtain IOH-NPs with a saline composition [ZrO]²⁺GMP]²⁻ (FIG. 1).

In detail, [ZrO]²⁺[GMP]²⁻ IOH-NPs were synthesized by injection of aqueous ZrOCl₂×8H2O into an aqueous solution of H₂(GMP) (FIG. 1a), which, after purification, results in colloidally highly stable suspensions (5 mg/mL) that do not show any sedimentation over several weeks (FIG. 1b). Particle size, size distribution and zeta-potential analysis were examined by dynamic light scattering (DLS) and scanning electron microscopy (SEM). Accordingly, a hydrodynamic diameter of 69±16 nm (DLS) and a diameter of 29±4 nm (SEM) were obtained with negative surface charging (−35±6 mV) (FIG. 1b,c,e), which is also causative for the colloidal stability. The chemical composition of the [ZrO]²⁺[GMP]²⁻ IOH-NPs was validated by different methods, including X-ray diffraction (XRD), energy-dispersive electron spectroscopy (EDXS), Fourier-transform infrared (FT-IR) spectroscopy, elemental analysis (EA, C/H/N/S analysis), and total organics combustion/thermogravimetry (TG) (FIG. 1d). Whereas the presence of zirconium and GMP were qualitatively confirmed by EDXS and FT-IR, the [ZrO]²⁺:[GMP]²⁻ ratio and the overall composition were quantified by EA and TG. In sum, the composition [ZrO]²⁺[GMP]²⁻ with a GMP-load of 76 % of the total IOH-NP mass (24 % due to [ZrO]²⁺ as inorganic cation) was confirmed.

Here, it should be noticed that IOH-NPs with gemcitabine triphosphate (GTP) as the drug anion ([ZrO]²⁺₃[GTP]³⁻₂ IOH-NPs) can be realized similarly, showing comparable properties as [ZrO]²⁺[GMP]²⁻ IOH-NPs.

In addition to [ZrO]²⁺[GMP]²⁻ IOH-NPs as active drug-loaded nanocarrier, [ZrO]²⁺[CMP]²⁻ IOH-NPs (CMP: cytidine monophosphate) were prepared as GEM-free reference via a similar synthesis protocol. In difference to GMP, CMP does not contain fluorine in 2,2′-position of the ribose unit, so that the DNA reproduction is not blocked. Beside the cytostatic activity, [ZrO]²⁺[GMP]²⁻ and [ZrO]²⁺[CMP]²⁻ IOH-NPs exhibit similar properties (size, composition, etc.) within the significance of the analytical characterization. Therefore, GEM-free [ZrO]²⁺[CMP]2- IOH-NPs were used (with similar concentration as [ZrO]²⁺[GMP]²⁻) as negative control without any cytostatic effect. Moreover, solutions of H₂(GMP) and free GEM were used (with similar GMP concentration as in [ZrO]²⁺[GMP]₂₋) as positive control.

### As a Summary of All Investigations Carried out With the Present Ioh-nps, the Following Has Been

revealed.

Efficient uptake of GMP-IOH-NPs has been shown into tumor cells in vitro. Fluorescence labelling of the IOH-NP allowed to assess their cell-uptake, biodistribution and tumor specific delivery. In this regard, multiscale imaging allows to the monitor biodistribution and tumor accumulation from in vivo via tissue to cellular levels by tracking the IOH-NPs, which is an essential key to understand the pharmacokinetic processes and to define an optimal application route.

Multiscale fluorescence microscopy confirmed the uptake of the IOH-NPs in tumor cells via endocytosis, followed by intracellular trafficking via endocytic pathways. Although the endosomal escape mechanisms for cytosol delivery of IOH-NPs or already released GMP within endosomal vesicles are not yet sufficiently defined, GMP is finally delivered from the endosomes/lysosomes to the cytosol as demonstrated by their antitumor efficacy. Notably, the uptake of GMP-IOH-NPs is independent of the activity of the human equilibrative nucleoside transporter (hENT1), which is responsible for the transport of free GEM into cells. As a result, IOH-NPs overcome the often occurring chemoresistance of GEM due to a downregulation of hENT1. Most probably, GMP-IOH-NPs are also independent of dCK for GEM activation since the GMP-IOH-NPs already contain phosphorylated GMP. In orthotopic PDAC mouse models, a high accumulation of GMP-IOH-NPs in primary tumor lesions as well as at metastatic sites is verified, which is highly beneficial in treating PDAC since more than half of the patients exhibit metastasis at the time of diagnosis. GMP-IOH-NPs are delivered to tumor cells, protecting GEM during the delivery process by preventing the metabolic inactivation of GEM by enzymes present in the circulation and liver. The treatment with GMP-IOH-NPs is well tolerated and results in a higher anti-tumor efficacy compared to free GEM, which was enhanced even further applying cetuximab-functionalized GMP-CTX-IOH-NPs.

By minimizing undesired side effects, overcoming chemoresistance, preventing GEM inactivation by delivering already phosphorylated GEM to tumor sites with GMP-IOH-NPs, we could address most of the disadvantageous associated with current GEM-based PDAC therapy. Associating the results to therapy efficacy will further support the design of the IOH-NPs as drug-delivery system with a high chance to further improve treatment efficacy. Together with maximizing therapeutic benefits by high drug load, advantageous biodistribution, characterized by almost no liver accumulation but tumor-specific delivery after intraperiotenal application, and the option of long-term GEM treatment by circumventing the mechanisms of chemoresistance, we anticipate GMP-IOH-NPs to have a high chance of improving the quzality of life and survival of PDAC patients.

### EXAMPLES

Synthesis of Inorganic-organic Hybrid Nanoparticles [ZrO]²⁺[GMP]²⁻IOH-NPs (designated as GMP-IOH-NPs in in vitro/in vivo studies). The synthesis of [ZrO]²⁺[GMP]²⁻ IOH-NPs is characterized by a straightforward water-based precipitation at room temperature. Accordingly, a concentrated aqueous solution of ZrOCl₂×8H2O was injected with vigorous stirring into an aqueous solution of H₂(GMP). Following the LaMer-Dinegar model of particle nucleation and particle growth, a high supersaturation is induced by the injection, which induces rapid nucleation, and thus, the formation of uniform nanoparticles.

For the synthesis of [ZrO]²⁺[GMP]²⁻ IOH-NPs, 100 μL of an aqueous solution of ZrOCl₂×8H₂O (14.6 mg, 45.5 μmol, 99.9 %, Sigma Aldrich, Germany) were injected into 10 mL of an aqueous solution of gemictabine monophosphate (GMP, 17.2 mg, 50 μmol, ≥97 %, Toronto Research Chemicals, Canada). After 2 min of intense stirring, the IOH-NPs were separated by centrifugation (25.000 rpm, 15 min) and twice purified by redispersion/centrifugation in/from H₂O. For further use, the [ZrO]²⁺[GMP]²⁻ IOH-NPs were dispersed in demineralized water or dried to powder samples. After purification and redispersion, colorless suspensions of [ZrO]²⁺[GMP]²⁻ were obtained, which are colloidally stable over several weeks.

[Zro]²⁺₃[GTP]³⁻₂ IOH-NPs (designated As GTP-IOH-NPs in in vitro studies). for the synthesis of [ZrO]²⁺₃[GTP]³⁻₂ IOH-NPs, 100 μL of an aqueous solution of ZrOCl₂×8H₂O (14.6 mg, 45.5 μmol, 99.9 %, Sigma Aldrich, Germany) were injected into 10 mL of an aqueous solution of sodium gemictabine triphosphate (GTP, 18.2 mg, 50 μmol, ≥95 %, Jena Biosciences, Germany). After 2 min of intense stirring, the IOH-NPs were separated by centrifugation (25.000 rpm, 15 min) and twice purified by redispersion/centrifugation in/from H₂O. For further use, the [ZrO]²⁺3[GTP]³⁻₂ IOH-NPs were dispersed in demineralized water or dried to powder samples. After purification and redispersion, colorless suspensions of [ZrO]²⁺₃[GTP]³⁻₂ IOH-NPs were obtained, which are colloidally stable over several weeks. [ZrO]²⁺[CMP]²⁻ IOH-NPs (designated as Ref-IOH-NPs in in vitro/in vivo studies). Since cytidine monophosphate (CMP, C₉H₁₂N₃O₈P) has a similar structure as gemcitabine monophosphate (GMP, C₉H₁₀N₃O₇F₂P) but without any therapeutic activity, [ZrO]²⁺[CMP]²⁻ IOH-NPs were prepared as negative-control nanoparticles for biological studies. Since GMP is very expensive but chemically similar to CMP, moreover, [ZrO]²⁺[CMP]²⁻ IOH-NPs were used to characterize many properties of the IOH-NPs. [ZrO]²⁺[CMP]2-IOH-NPs were prepared by dissolving 36.7 mg (0.1 mmol) of cytidine monophosphate sodium salt (Na₂(CMP), ≥97 %, Biosynthesis, United Kingdom) in 50 mL of demineralized water. Thereafter, 0.5 mL of an aqueous solution containing 29.3 mg (0.09 mmol) of ZrOCl₂×8H₂O (99.9 %, Sigma Aldrich, Germany) were injected, which results in an instantaneous nucleation of IOH-NPs. After 2 min of intense stirring, the as-prepared IOH-NPs were separated via centrifugation (25.000 rpm, 15 min) and twice purified by redispersion/centrifugation in/from H₂O. For further use, the [ZrO]²⁺[CMP]²⁻ IOH-NPs were dispersed in demineralized water or dried to powder samples.

Fluorescence labelling. For biological studies on cellular uptake and efficacy, the IOH-NPs were fluorescence-labelled by addition of small amounts of the fluorescent dye DY™-549P1-dUTP (DUT549, Dyomics, Germany) for in vitro studies and the fluorescent dye DY™-647P1-dUTP (DUT647, Dyomics, Germany) for in vivo studies. To this concern, 25 nmol of DUT549 or DUT647 were added to the respective solution of [GMP]²⁻ or [CMP]²⁻ prior to initiating the nanoparticle nucleation by the injection of the ZrOCl₂×8H₂O solutions. In this way, fluorescent IOH-NPs with the composition [ZrO]²⁺[(CMP)_{>0.99}(DUT)_{<0.01}]²⁻ or [ZrO]²⁺[(GMP)_{>0.99}(DUT)_{<0.01}]²⁻ were formed. The fluorescence-labelled IOH-NPs show a pink (DUT549) or blue (DUT647) color, which is characteristic for the respective DUT dye.

Surface functionalization by glucose (designated as GMP-GLU-IOH-NPs in in vitro/in vivo studies). Surface functionalization with glucose was performed by coating the IOH-NPs with a layer of [ZrO]²⁺[G6P]²⁻ (G6P: glucose-6-phosphate, C₆H₁₁O₉P). To this concern, [ZrO]²⁺[GMP]²⁻ or [ZrO]²⁺[CMP]²⁻ IOH-NPs were prepared as described. Subsequent to synthesis, they exhibit a negative surface charge. In the following, an aqueous solution of 1 mL of ZrOCl₂×8H₂O (10.8 mg, 33.8μmol, 99.9 %, Sigma Aldrich, Germany) was added to the [ZrO]²⁺[GMP]²⁻/[ZrO]²⁺[CMP]²⁻IOH-NP suspension and stirred for 1 h at room temperature. Thereafter, the suspension was centrifuged (25.000 rpm, 15 min) and redispersed to remove excess [ZrO]²⁺, which could promote undesired additional particle nucleation instead of a growth of the preformed nanoparticles. Finally, 1 mL of an aqueous Na₂(G6P) solution (10.6 mg, 37.5 μmol, Sigma Aldrich, Germany) was added slowly at a defined flow rate (1 mL/h) using a syringe pump. The resulting nanoparticles were separated via centrifugation (25.000 rpm, 15 min) and purified by redispersion/centrifugation in/from H₂O. For analytical characterization, the [ZrO]²⁺[GMP]²⁻@[ZrO]²⁺[G6P]²⁻ or [ZrO]²⁺[CMP]2-@[ZrO]²⁺[G6P]2-IOH-NPs IOH-NPs were dispersed in demineralized water or dried to powder samples.

Surface Functionalization With Cetuximab As Antibody (designated As GMP-CTX-IOH-NPs in in vitro/in vivo studies). The antibody cetuximab (CTX, Erbitux®, solution for infusion, Merck, Germany) was used to functionalize the surface of the IOH-NPs by electrostatic adsorption. CTX at a concentration range of 0.1-0.5 mg/mL was added to an aqueous suspension of [ZrO]²⁺[GMP]²⁻ or [ZrO]²⁺[CMP]²⁻ IOH-NPs (1 mg/mL) and stirred for 1 h at room temperature. Thereafter, the IOH-NPs were separated by centrifugation. The CTX-functionalized IOH-NPs were resuspended in demineralized water for further characterization, while the supernatant after centrifugation was collected to measure the antibody concentration. Bradford assays were used to quantify the CTX concentration in the supernatant (Bradford reagent, Sigma Aldrich, Germany). The amount of the antibody on the IOH-NP surface was calculated by the initial amount minus the amount detected in the supernatant.

Subsequent to the surface functionalization, the ctx-functionalized IOH-NPs were collected by centrifugation and resuspended in water. The supernatant was separated and the microprotocol (1-10 μg/mL protein) of the Bradford assay performed to measure the antibody concentration. The amount of CTX was adapted so that it was just detectable in the supernatant. As a result, a maximum amount of CTX was adsorbed on the IOH-NP surface. To this concern, 1 mL of the Bradford reagent (Sigma Aldrich, Germany) was mixed to 1 mL of the supernatant after CTX treatment and incubated for 15 minutes. Subsequently, the absorbance was measured at 595 nm with a UV 2700 spectrophotometer (Shimadzu, Japan). Finally, a calibration curve was used to determine the CTX amount.

Immunofluorescence assay. For the indirect fluorescence immunoassay, the as-prepared IOH-NPs were immobilized by passive adsorption at the bottom of a microtiter plate containing MaxiSorp (Thermo Fisher Scientific, Germany). To this concern, CTX-functionalized IOH-NP and the free CTX antibody control group were diluted in 50 mM of carbonate-bicarbonate buffer (pH=9.6) at different concentrations (1.2-600μg/mL), under the assumption that 2 mg of IOH-NPs bear 2.8 μmol CTX on the particle surface. 200 μL of the dilutions were transferred in each well and incubated overnight at 4° C. Then, the supernatant was decanted and the wells were washed two times with 200 μL PBS washing buffer (137 mM NaCl, 2.7 mM KCl, 10 mM Na₂HPO₄, KH₂PO₄, 0.05 % Tween, pH=7.4). The wells were then blocked with 200 μL 5% BSA-solution in PBS buffer for 2 h and washed again two times with PBS washing buffer. Finally, 100 μL of the FITC-labelled (FITC: fluorescein isothiocyanate) rabbit anti-human IgG antibody was added as the secondary antibody with a concentration of 2.5 μg/mL. After an incubation time of 1 h, the wells were washed four times with 200 μL of PBS washing buffer. Fluorescence measurements were performed on a Synergy H1 Hybrid Reader (Agilent Biotek, Germany) at an excitation wavelength of 490 nm and an emission wavelength of 530 nm.

In addition, the fluorescence immunoassay was extended to determine the biological activity in terms of receptor recognition of the antibody on the IOH-NP surface. For this purpose, the microtiter plate was first coated with streptavidin (STV) to subsequently immobilize the biotinylated hEGFR1. For this, 50 μL of a 200 nM STV solution in PBS buffer (137 mM NaCl, 2.7 mM KCl, 10 mM Na₂HPO₄, KH₂PO₄, pH=7.4) was added to the wells of a microtiter plate and incubated at 4° C. on for 72 hours. Subsequently, each well of the microtiter plate was washed three times with 240 μL of TBS buffer (20 mM Tris-Base, 150 mM NaCl, pH=7.35) and incubated with 200 μL of a 5 % BSA-solution in PBS buffer for 2 h at 4° C. Then, the wells were washed three times with PBS washing buffer and 50 μL of biotinylated hEGFR1 was added in each well at a concentration of 2 μg/mL for receptor immobilisation through biotin-streptavidin binding. After 2 h incubation time, the wells were washed two times with TETBS buffer (20 mM Tris-Base, 150 mM NaCl, 5 mM EDTA, 0.05 % (v/v) Tween-20, pH=7.35) supplemented with 800 μM d-biotin to block free STV binding sites. After a second washing step with PBS washing buffer, 200 μL of CTX-functionalized IOH-NPs and the control groups (free CTX and Trastuzumab) were added in the wells and all parameters from the assay description above were retained.

Cytotoxic effects of GMP-NP on GEM resistant murine KPC cells and GMP-NP or GTP-NP on GEM resistant human AsPC-1 pancreatic cancer cells in comparison to control KPC and AsPC-1 pancreatic cancer cells.

In the course of the present invention, the inventors have assessed the cytotoxicity of gemcitabine monophosphate containing nanoparticles, GMP-IOH-NPs on GEM resistant murine KPC pancreatic cancer cells and the cytotoxicity of GMP-IOH-NPs and gemcitabine triphosphate containing nanoparticles, GTP-IOH-NPs, on human GEM resistant AsPC-1 pancreatic cancer cells, all in comparison to the treatment with free GEM.

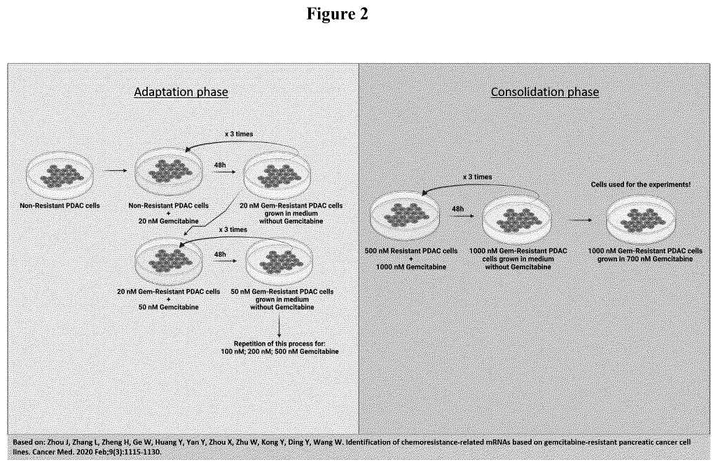

In order to study the anti-tumor efficacy of GMP-IOH-NPs and GTP-IOH-NPs in comparison to the free GEM drug on GEM-chemo-resistant PDAC cells, the GEM-resistant murine PDAC cell line KPC and the GEM-resistant human PDAC cell line AsPC-1 were established using a two-step method adapted from Zhou J, Zhang L, Zheng H, et al. Identification of chemoresistance-related mRNAs based on gemcitabine-resistant pancreatic cancer cell lines. Cancer Med. 2020; 9:1115-1130. PMID: 31823522, as illustrated in FIG. 2.

In the adaptation stage, KPC or AsPC-1 cells were treated with GEM for 48 hours, with stepwise increases in concentration from 20 to 500 nM (20, 50, 100, 200 and 500 nM). After each dose-induced step, apoptotic cells were discarded, and surviving cells were expanded in GEM-free culture medium. This process was repeated three times. In the consolidation stage, cells were then exposed to a final GEM concentration of 1000 nM. Following the last round of treatment at this concentration, KPC as well as AsPC-1 cells acquired resistance to GEM. To maintain the GEM-resistant phenotype, cells were cultured in medium containing 700 nM GEM.

The native KPC cell line was provided by Prof. Volker Ellenrieder (Clinic for Gastroenterology, Gastrointestinal Oncology and Endocrinology, University Medical Center Göttingen, Germany). These cells were derived from KPC mice (Hingorani et al., 2005). KPC cells were cultured in Dulbecco's Modified Eagle Medium (DMEM; GIBCO) supplemented with 10 % fetal bovine serum (FBS; GIBCO), 1 % penicillin and streptomycin (pen/strep), and 1 % L-glutamine. Cells were cultivated at 37° C. under a humidified atmosphere of 5 % CO₂.

The human AsPC-1 PDAC cell line was obtained from ATCC, USA (CRL-1682) and grown in RPMI 1640medium supplemented with 10 % fetal bovine serum (FBS) and GlutaMAX (Gibco, USA), 1 % penicillin and streptomycin (pen/strep) as described (Saccomano et al., 2016). Cells were cultivated at 37° C. under a humidified atmosphere of 5 % CO₂.

CellTiter-Glo® luminescent viability assay: KPC and AsPC-1 native cells (control) and KPC-GEM and AsPC-1-GEM resistant cells were seeded in a 96-well plate at a concentration of 10,000 cells per well. After overnight incubation, a single dose of increasing concentrations (1000 nM-2000 nM) of free GEM, or [ZrO]²⁺[CMP]²⁻ IOH-NPs (CMP: cytidine monophosphate) used as GEM-free reference (Ref-IOH-NP), or gemcitabine monophosphate (GMP) containing GMP-IOH-NPs or gemcitabine triphosphate (GTP) containing GTP-IOH-NPs were added to the cells at the start of the experiment. GMP-IOH-NPs have a composition [ZrO]²⁺[GMP]²⁻ with GMP as drug anion (76 % of total IOH-NP mass). GTP-IOH-NPs have a composition [ZrO]²⁺₃[GTP]³⁻₂ with GTP as drug anion (75 % of total IOH-NP mass). The efficacy of the free chemotherapeutic drug gemcitabine or of the nanoparticles was normalized to an /stimulated/ untreated condition. Cell viability was assessed using the CellTiter-Glo assay (Promega, G8461). Following six days of drug treatment, after the supernatant aspiration, 80 μL of CellTiter-Glo reagent diluted 1:5 with phosphate buffered saline (PBS) was added to each well. The plate was shaken for 2 minutes at room temperature to facilitate cell lysis, then incubated for an additional 10 minutes without shaking to stabilize the luminescence signal. Luminescence was measured using a CLARIOstar multimode plate reader (BMG LABTECH, Germany).

### Results

Cytotoxic effects of GEM-NP on GEM resistant murine KPC cells in comparison to control KPC pancreatic cancer cells

To test the cytotoxic effect of GMP nanoparticles, GMP-IOH-NPs, on GEM-resistant cells, murine PDAC KPC cells were made resistant through dose-escalation GEM exposure.

To evaluate the cytotoxic effects of GMP-IOH-NPs on GEM-resistant KPC cells, both GEM-resistant and native KPC cells were incubated with increasing concentrations of GMP-IOH-NPs (1000 nM-2000 nM). The anti-tumor efficacy of these nanoparticles was compared to Ref-IOH-NP (negative control) and free GEM (positive control). The concentration-dependent efficacy on cell viability was measured as the percentage of live cells after 6 days of incubation by the CellTiter-Glo® luminescent viability assay and normalized to unstimulated, untreated KPC cells.

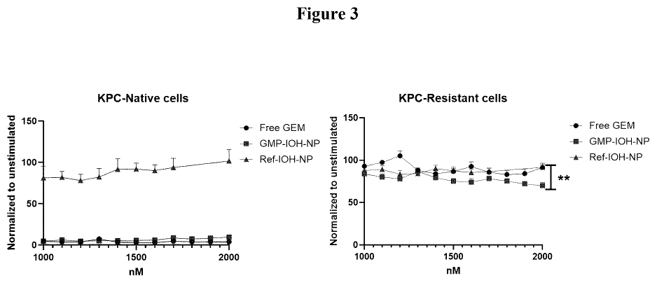

The sensitivity of GEM to treat native KPC cells is shown in FIG. 3, left graph. High doses of GMP-IOH-NPs and free GEM, starting from 1000 nM, resulted in complete cell death of native KPC cells, indicating sensitivity of KPC native cells to both treatments.

The non-toxic effect of GEM in the resistant KPC cells was confirmed by their maintained viability in the presence of high doses of free GEM (FIG. 3, right graph). The viability of KPC cells remained unchanged with Ref-IOH-NP treatment, even at concentrations up to 2000 nM, indicating that these nanoparticles are non-toxic. However, we observed a statistically significant 20 % reduction in the viability of GEM-resistant KPC cells in response to treatment with 2000 nM GMP-IOH-NPs, compared to those treated with 2000 nM free GEM (FIG. 3, right graph).

These results demonstrate the advantage of GMP-IOH-NPs in the efficacy to treat GEM resistant murine KPC cells in comparison to no effect by applying standard free GEM therapy most likely by circumventing gemcitabine chemoresistance mechanisms in KPC PDAC cells (FIG. 3, right graph).

Cytotoxic effects of GEM-NP or GTP-NP on human AsPC-1 pancreatic cancer cells in comparison to control AsPC-1 pancreatic cancer cells

To test the cytotoxic effect of GMP/GTP nanoparticles, GMP-IOH-NPs and GTP-IOH-NPs, on GEM-resistant cells, human PDAC-AsPC-1 cells were made resistant through dose-escalation GEM exposure.

To evaluate the cytotoxic effects of GMP-IOH-NPs as well as GTP-IOH-NPs on GEM-resistant human AsPC-1 cells, both GEM-resistant and native AsPC-1 cells (controls) were incubated with increasing concentrations of GMP-IOH-NPs or GTP-IOH-NPs (1000 nM-2000 nM). The anti-tumor efficacy of these nanoparticles was compared to free GEM (positive control). The concentration-dependent efficacy on cell viability was measured as the percentage of live cells after 6 days of incubation by the CellTiter-Glo® luminescent viability assay and normalized to non-treated (unstimulated) AsPC-1 cells.

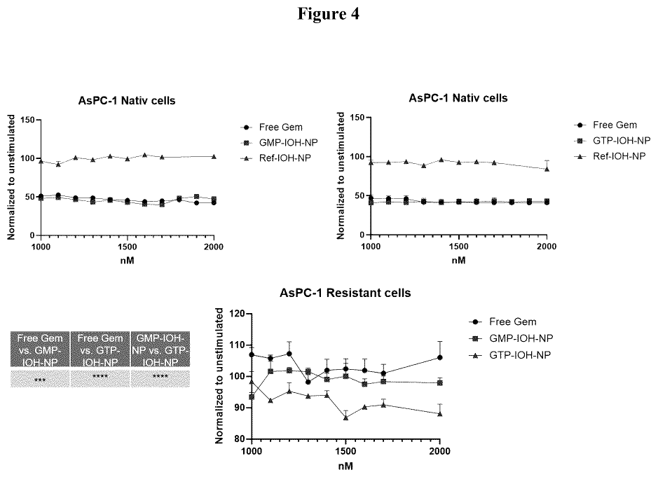

The sensitivity of GEM or GMP-IOH-NPs to treat native AsPC-1 cells is shown in FIG. 4, upper graph, left panel, and the sensitivity of GEM or GTP-IOH-NPs to treat native AsPC-1 cells in FIG. 4, upper graph, right panel. The viability of AsPC-1 native cells remained unchanged with Ref-IOH-NP treatment, even at concentrations up to 2000 nM, indicating that these nanoparticles are non-toxic. High doses of GMP-IOH-NPs, GTP-IOH-NPs and free GEM, starting from 1000 nM, resulted in ˜50 % cell death of native AsPC-1 cells, indicating sensitivity of AsPC-1 native cells to these treatments (GMP-IOH-NPs, GTP-IOH-NPs and free GEM), and also demonstrating that AsPC-1 cells provide already GEM resistance to a certain amount under these conditions.

The non-toxic effect of GEM in the resistant AsPC-1 cells was confirmed by their maintained viability in the presence of high doses of free GEM (FIG. 4, lower graph, blue line). However, the inventors observed a statistically significant reduction in the viability of GEM-resistant AsPC-1 cells in response to different concentrations of treatment with GMP-IOH-NPs in the range of 1000 - 2000 nM (p≤0.001) and even more pronounced in response to different concentrations of GTP-IOH-NPs in the range of 1000-2000 nM (p≤0.0001), compared to those treated with free GEM in the range of 1000-2000 nM (FIG. 4, lower panel).

These results demonstrate the advantage of GMP-IOH-NPs in the efficacy to treat GEM resistant AsPC-1 cells, and show that GTP-IOH-NPs is even more potent, in comparison to no cytotoxic effect by applying standard free GEM therapy most likely by circumventing gemcitabine chemoresistance mechanisms in AsPC-1 cells (FIG. 4, lower panel).

In particular, the study demonstrates that GMP-IOH-NPs are still cytotoxic in GEM-resistant murine KPC cells in which free GEM application does not have any effect on tumor cell death anymore. Treatment with 2000 nM GMP-IOH-NPs resulted in a statistically significant 20% reduction in cell viability of GEM-resistant KPC cells compared to no effect in response to 2000 nM free GEM, underscoring the superior effectiveness of GMP-IOH-NPs to treat chemoresistant PDAC cells compared to free GEM by overcoming GEM resistance.

Furthermore, the study demonstrates that GMP-IOH-NPs and GTP-IOH-NPs (even more potent) are still cytotoxic in GEM-resistant AsPC-1 cells in which free GEM application does not have any effect on tumor cell death anymore. Treatment with different concentrations of GMP-IOH-NPs in the range of 1000 to 2000 nM resulted in a reduction in cell viability of GEM-resistant AsPC-1 cells compared to no effect in response to free GEM. This cytotoxic effect was even more potent when applying GTP-IOH-NPs in the range of 1000 to 2000 nM to GEM-resistant AsPC-1 cells underscoring the superior effectiveness of GMP-IOH-NPs and even more GTP-IOH-NPs to treat GEM-resistant PDAC cells compared to free GEM by overcoming GEM resistance.

Since the therapeutic efficacy of gemcitabine is limited by the innate and acquired resistance leading to treatment failure and recurrent disease in most PDAC patients, this novel drug-delivery system by GMP-IOH-NPs or GTP-IOH-NPs has a high chance to significantly improve current PDAC treatment and increase clinical responses of this deadly disease as well as patient outcome not only by selective delivery of extraordinarily high concentrations of already phosphorylated gemcitabine monophosphate (GMP) or gemcitabine triphosphate (GTP) to the primary tumor and metastatic sites and by reduced side effects, but also by being still cytotoxic in already GEM resistant PDAC cells or cells that have become resistant during GEM therapy, by circumventing chemoresistance mechanisms.

To sum up, GEM-IOH-NPs can overcome the often occurring chemoresistance of GEM in PDAC cells since
the encapsulation of already phosphorylated gemcitabine in nanoparticles as GMP-IOH-NPs or as GTP-IOH-NPs enables intracellular GEM activation independent of the deoxycytidine kinase (dCK), an enzyme often inactivated in GEM-resistant PDAC cells, thereby circumventing GEM toxicity (Dash, et al 2024), and
GMP-IOH-NPs and GTP-IOH-NPs are taken up by the tumor cells most likely via one of the endocytic pathways, independent of the activity of the human equilibrative nucleoside transporter (hENT1), which is responsible for the transport of free GEM into cells and often downregulated due to chemoresistance.

1. An inorganic-organic hybrid compound as ionic compound, comprising the cation [ZrO]²⁺ and an organic active ingredient anion selected from gemcitabine monophosphate or gemcitabine triphosphate.

2. The inorganic-organic hybrid compound as claimed in claim 1, which has a particle diameter in the range from 1 to 100 nm.

3. The inorganic-organic hybrid compound as claimed in claim 1, said compound being [ZrO]²⁺[GMP]²⁻ or [ZrO]²⁺₃[GTP]³⁻₂.

4. The inorganic-organic hybrid compound as claimed in claim 1, which is in X-ray-amorphous form.

5. The inorganic-organic hybrid compound as claimed in claim 1, further comprising a fluorescent dye anion which carries a phosphate, phosphonate, sulfate, sulfonate, carbonate, or carboxylate group as functional group.

6. The inorganic-organic hybrid compound as claimed in claim 5, the organic fluorescent dye anion being derived from fluorescent dyes selected from the group consisting of 1,1′-diethyl-2,2′-cyanine iodide, 1,2-diphenylacetylene, 1,4-diphenylbutadiene, 1,6-diphenylhexatriene, 2,5-diphenyloxazole, 2-methylbenzoxazole, 4′,6-diamidino-2-phenylindole (DAPI), 4-(dicyanomethylene)-2-methyl-6-(p-dimethylaminostyryl)-4H-pyran (DCM), 4-dimethylamino-4′-nitrostilbene, 5,10,15-triphenylcorrole, 5,10,15-tris(pentafluorophenyl)corrole, 5,10-diarylchlorin, 5,10-diarylcopper chlorin, 5,10-diarylcopper oxochlorin, 5,10-diarylmagnesium oxochlorin, 5,10-diaryloxochlorin, 5,10-diarylzinc chlorin, 5,10-diarylzinc oxochlorin, 7-benzylamino-4-nitrobenz-2-oxa-1,3-diazole, 7-methoxycoumarin-4-acetic acid, 9,10-bis(phenylethynyl)anthracene, 9,10-diphenylanthracene, acridine orange, acridine yellow, adenine, anthracene, anthraquinone, auramine O, azobenzene, bacteriochlorophyll A, benzoquinone, beta-carotene, bilirubin, biliverdin dimethyl ester, biphenyl, bis(5-mesityldipyrrinato)zinc, bis(5-phenyldipyrrinato)zinc, boron subphthalocyanine chloride, chlorin E6, chlorophyll A, chlorophyll B, cis-stilbene, coumarin and its derivatives, cresyl violet perchlorate, cryptocyanine, crystal violet, cytosine, dansylglycine, diprotonated tetraphenylporphyrin, eosine and its derivatives, ethyl (p-dimethylamino)benzoate, ferrocene, fluorescein and its derivatives, as for example methylfluorescein, resorufin, amaranth, aluminum(III)-phthalocyanine chloride tetrasulfonic acid, trypan blue, guanine, hematin, histidine, Hoechst 33258, indocarbocyanine and its derivatives, lucifer yellow CH, magnesium octaethylporphyrin, magnesium phthalocyanine, magnesium tetramesitylporphyrin, magnesium tetraphenylporphyrin, malachite green, merocyanine, N, N′-difluoroboryl-1,9-dimethyl-5-(4-iodophenyl)dipyrrin, N, N′-difluoroboryl-1,9-dimethyl-5-[(4-(2-trimethylsilylethynyl), N,N′-difluoroboryl-1,9-dimethyl-5-phenyldipyrrin, tetraphenylporphyrin, naphthalene, nile blue, nile red, octaethylporphyrin, oxacarbocyanine and its derivatives, oxazine and its derivatives, p-quaterphenyl, p-terphenyl, perylene and its derivatives, phenol, phenylalanine, phenyldipyrrin, pheophorbide, phthalocyanine, pinacyanol iodide, piroxicam, porphin, proflavin, protoporphyrin IX dimethyl ester, pyrene, pyropheophorbide and its derivatives, pyrrol, quinine, rhodamine and its derivatives, riboflavin, bengal red, squarylium dye III, TBP beta-octa(COOBu)-Fb, TBP beta-octa(COOBu)-Pd, TBP beta-octa(COOBu)-Zn, TBP meso-tetraphenyl-beta-octa(COOMe)-Fb, TBP meso-tetraphenyl-beta-octa(COOMe)-Pd, TBP meso-tetraphenyl-beta-octa(COOMe)-Zn, TCPH meso-tetra(4-COOMe-phenyl)-Fb, TCPH meso-tetra(4-COOMe-phenyl)-Pd, TCPH meso-tetra(4-COOMe-phenyl)-Zn, tetra-tert-butylazaporphin, tetra-tert-butylnaphthalocyanine, tetrakis(2,6-dichlorophenyl)porphyrin, tetrakis(o-aminophenyl)porphyrin, tetramesitylporphyrin, tetraphenylporphyrin, tetraphenylsapphyrin, thiacarbocyanine and its derivatives, thymine, trans-stilbene, tris(2,2′-bipyridyl)ruthenium(II), tryptophan, thyrosine, uracil, vitamin B12, zinc octaethylporphyrin, phthalocyanine and its derivatives, porphyrin and its derivatives, including tetra(o-amidophosphonatophenyl)porphyrin, and umbelliferone, where the organic fluorescent dyes which do not as such have a phosphate, phosphonate, sulfate, sulfonate, carbonate, or carboxylate group have been modified with at least one of these functional groups.

7. The inorganic-organic hybrid compound as claimed in claim 1, which is doped with a lanthanoid selected from Ce, Pr, Nd, Sm, Eu, Gd, Tb, Dy, Ho, Er, Tm, Yb, or Lu, with a transition metal selected from Cr, Mn, Cu, Zn, Y, Ag, or Cd, with a main group element selected from Sn, Sb, Pb, or Bi, or with a complex anion selected from [VO₄]³⁻, [MoO₄]³⁻ or [WO₄]³⁻.

8. The inorganic-organic hybrid compound as claimed in claim 1, which is further functionalized with an antibody, peptide or oligonucleotide.

9. The inorganic-organic hybrid compound as claimed in claim 1, which is cetuximab-functionalized.

10. The inorganic-organic hybrid compound as claimed in claim 1, which is glucose-coated.

11. A method of treating pancreatic ductal adenocarcinoma (PDAC) by administering the inorganic-organic hybrid compound of claim 1.

12. The method of claim 11, wherein the inorganic-organic hybrid compound has a particle diameter in the range from 1 to 100 nm.

13. The method of claim 11, wherein the inorganic-organic hybrid compound is [ZrO]²⁺[GMP]²⁻ or [ZrO]²⁺₃[GTP]³⁻₂.

14. The method of claim 11, wherein the inorganic-organic hybrid compound is in X-ray-amorphous form.

15. The method of claim 11, wherein the inorganic-organic hybrid compound further comprises a fluorescent dye anion which carries a phosphate, phosphonate, sulfate, sulfonate, carbonate, or carboxylate group as functional group.

16. The method of claim 15, wherein the fluorescent dye anion is derived from fluorescent dyes selected from the group consisting of 1,1′-diethyl-2,2′-cyanine iodide, 1,2-diphenylacetylene, 1,4-diphenylbutadiene, 1,6-diphenylhexatriene, 2,5-diphenyloxazole, 2-methylbenzoxazole, 4′,6-diamidino-2-phenylindole (DAPI), 4-(dicyanomethylene)-2-methyl-6-(p-dimethylaminostyryl)-4H-pyran (DCM), 4-dimethylamino-4′-nitrostilbene, 5,10,15-triphenylcorrole, 5,10,15-tris(pentafluorophenyl)corrole, 5,10-diarylchlorin, 5,10-diarylcopper chlorin, 5,10-diarylcopper oxochlorin, 5,10-diarylmagnesium oxochlorin, 5,10-diaryloxochlorin, 5,10-diarylzinc chlorin, 5,10-diarylzinc oxochlorin, 7-benzylamino-4-nitrobenz-2-oxa-1,3-diazole, 7-methoxycoumarin-4-acetic acid, 9,10-bis(phenylethynyl)anthracene, 9,10-diphenylanthracene, acridine orange, acridine yellow, adenine, anthracene, anthraquinone, auramine O, azobenzene, bacteriochlorophyll A, benzoquinone, beta-carotene, bilirubin, biliverdin dimethyl ester, biphenyl, bis(5-mesityldipyrrinato)zinc, bis(5-phenyldipyrrinato)zinc, boron subphthalocyanine chloride, chlorin E6, chlorophyll A, chlorophyll B, cis-stilbene, coumarin and its derivatives, cresyl violet perchlorate, cryptocyanine, crystal violet, cytosine, dansylglycine, diprotonated tetraphenylporphyrin, eosine and its derivatives, ethyl (p-dimethylamino)benzoate, ferrocene, fluorescein and its derivatives, as for example methylfluorescein, resorufin, amaranth, aluminum(III)-phthalocyanine chloride tetrasulfonic acid, trypan blue, guanine, hematin, histidine, Hoechst 33258, indocarbocyanine and its derivatives, lucifer yellow CH, magnesium octaethylporphyrin, magnesium phthalocyanine, magnesium tetramesitylporphyrin, magnesium tetraphenylporphyrin, malachite green, merocyanine, N, N′-difluoroboryl-1,9-dimethyl-5-(4-iodophenyl)dipyrrin, N,N′-difluoroboryl-1,9-dimethyl-5-[(4-(2-trimethylsilylethynyl), N,N′-difluoroboryl-1,9-dimethyl-5-phenyldipyrrin, tetraphenylporphyrin, naphthalene, nile blue, nile red, octaethylporphyrin, oxacarbocyanine and its derivatives, oxazine and its derivatives, p-quaterphenyl, p-terphenyl, perylene and its derivatives, phenol, phenylalanine, phenyldipyrrin, pheophorbide, phthalocyanine, pinacyanol iodide, piroxicam, porphin, proflavin, protoporphyrin IX dimethyl ester, pyrene, pyropheophorbide and its derivatives, pyrrol, quinine, rhodamine and its derivatives, riboflavin, bengal red, squarylium dye III, TBP beta-octa(COOBu)-Fb, TBP beta-octa(COOBu)-Pd, TBP beta-octa(COOBu)-Zn, TBP meso-tetraphenyl-beta-octa(COOMe)-Fb, TBP meso-tetraphenyl-beta-octa(COOMe)-Pd, TBP meso-tetraphenyl-beta-octa(COOMe)-Zn, TCPH meso-tetra(4-COOMe-phenyl)-Fb, TCPH meso-tetra(4-COOMe-phenyl)-Pd, TCPH meso-tetra(4-COOMe-phenyl)-Zn, tetra-tert-butylazaporphin, tetra-tert-butylnaphthalocyanine, tetrakis(2,6-dichlorophenyl)porphyrin, tetrakis(o-aminophenyl)porphyrin, tetramesitylporphyrin, tetraphenylporphyrin, tetraphenylsapphyrin, thiacarbocyanine and its derivatives, thymine, trans-stilbene, tris(2,2′-bipyridyl)ruthenium(II), tryptophan, thyrosine, uracil, vitamin B12, zinc octaethylporphyrin, phthalocyanine and its derivatives, porphyrin and its derivatives, including tetra(o-amidophosphonatophenyl)porphyrin, and umbelliferone, where the organic fluorescent dyes which do not as such have a phosphate, phosphonate, sulfate, sulfonate, carbonate, or carboxylate group have been modified with at least one of these functional groups.

17. The method of claim 11, wherein the inorganic-organic hybrid compound is doped with a lanthanoid selected from Ce, Pr, Nd, Sm, Eu, Gd, Tb, Dy, Ho, Er, Tm, Yb, or Lu, with a transition metal selected from Cr, Mn, Cu, Zn, Y, Ag, or Cd, with a main group element selected from Sn, Sb, Pb, or Bi, or with a complex anion selected from [VO₄]³⁻, [MoO4]³⁻ or [WO₄]³⁻.

18. The method of claim 11, wherein the inorganic-organic hybrid compound is further functionalized with an antibody, peptide, or oligonucleotide.

19. The method of claim 11, wherein the inorganic-organic hybrid compound is cetuximab-functionalized.

20. The method of claim 11, wherein the inorganic-organic hybrid compound is glucose-coated.
